SEQUENCE LISTING (8 sequences)

SEQ ID NO: 1 - DNA, 2515 bp, synthetic construct
  source 1..2515: mol_type = other DNA
ttagatcaaataacccgtcccgaaatccaatatatatatatatataatattcaaactctctctttctatcttcgtacagtttaatagaagtaataatgggtaagaattacaagtccctggactctgttgtggcctctgacttcatagccctaggtatcacctctgaagttgctgagacactccatggtagactggccgagatcgtgtgtaattatggcgctgccactccccaaacatggatcaatattgccaaccatattctgtcgcctgacctccccttctccctgcaccagatgctcttctatggttgctataaagactttggacctgcccctcctgcttggatacccgacccggagaaagtaaagtccaccaatctgggcgcacttttggagaagcgaggaaaagagtttttgggagtcaagtataaggatcccatttcaagcttttctcatttccaagaattttctgtaagaaaccctgaggtgtattggagaacagtactaatggatgagatgaagataagtttttcaaaggatccagaatgtatattgcgtagagatgatattaataatccagggggtagtgaatggcttccaggaggttatcttaactcagcaaagaattgcttgaatgtaaatagtaacaagaaattgaatgatacaatgattgtatggcgtgatgaaggaaatgatgatttgcctctaaacaaattgacacttgaccaattgcgtaaacgtgtttggttagttggttatgcacttgaagaaatgggtttggagaagggttgtgcaattgcaattgatatgccaatgcatgtggatgctgtggttatctatctagctattgttcttgcgggatatgtagttgtttctattgctgatagtttttctgctcctgaaatatcaacaagacttcgactatcaaaagcaaaagccatttttacacaggatcatattattcgtgggaagaagcgtattcccttatacagtagagttgtggaagccaagtctcccatggccattgttattccttgtagtggctctaatattggtgcagaattgcgtgatggcgatatttcttgggattactttctagaaagagcaaaagagtttaaaaattgtgaatttactgctagagaacaaccagttgatgcctatacaaacatcctcttctcatctggaacaacaggggagccaaaggcaattccatggactcaagcaactcctttaaaagcagctgcagatgggtggagccatttggacattaggaaaggtgatgtcattgtttggcccactaatcttggttggatgatgggtccttggctggtctatgcttcactccttaatggggcttctattgccttgtataatggatcaccacttgtttctggctttgccaaatttgtgcaggatgctaaagtaacaatgctaggtgtggtccctagtattgttcgatcatggaaaagtaccaattgtgttagtggctatgattggtccaccatccgttgcttttcctcttctggtgaagcatctaatgtagatgaatacctatggttgatggggagagcaaactacaagcctgttatcgaaatgtgtggtggcacagaaattggtggtgcattttctgctggctctttcttacaagctcaatcattatcttcatttagttcacaatgtatgggttgcactttatacatacttgacaagaatggttatccaatgcctaaaaacaaaccaggaattggtgaattagcgcttggtccagtcatgtttggagcatcgaagactctgttgaatggtaatcaccatgatgtttattttaagggaatgcctacattgaatggagaggttttaaggaggcatggggacatttttgagcttacatctaatggttattatcatgcacatggtcgtgcagatgatacaatgaatattggaggcatcaagattagttccatagagattgaacgagtttgtaatgaagttgatgacagagttttcgagacaactgctattggagtgccacctttgggcggtggacctgagcaattagtaattttctttgtattaaaagattcaaatgatacaactattgacttaaatcaattgaggttatctttcaacttgggtttacagaagaaactaaatcctctgttcaaggtcactcgtgttgtgcctctttcatcacttccgagaacagcaaccaacaagatcatgagaagggttttgcgccagcaattttctcactttgaatgaagtgttataatataatacatacatatatgctttgaataaagtgtagttgattacatagagcagtttgcattatcaaatgcactaataataagttaaatgctctaaaatatgtgttaaatcttatggcaggattatagatttgattaagatagtcatattgaaccataaattttcattcaacacatttttagtgcatttgacattatttttattgaatttaataaatactgactacattgtatagtccactacatcat

SEQ ID NO: 2 - DNA, 1998 bp, synthetic construct
  source 1..1998: mol_type = other DNA
cacgagggtcacctacattcatccaaataacaggtctctgttctattctccaatccaatggagaaatctgggtatggaagagacggtatttacaggtctctgagaccacctctacacctccccaacaacaacaacctctcaatggtttcattccttttcagaaactcatcttcatacccacaaaagccagctctcattgattccgaaaccaaccaaatactctccttttcccacttcaaatctacggttatcaaggtctcccatggctttctcaatctgggtatcaagaaaaacgacgtcgttctcatctacgcccctaattctatccacttccctgtttgtttcctgggaattatagcctctggagccattgccactacctcaaatcctctctacacagtttccgagctttccaaacaggtcaaggattccaatcccaaactcattatcaccgttcctcaactcttggaaaaagtaaagggtttcaatctccccacgattctaattggtcctgattctgaacaagaatcttctagtgataaagtaatgacctttaacgatttggtcaacttaggtgggtcgtctggctcagaatttccaattgttgatgattttaagcagagtgacactgctgcgctattgtactcatctggcacaacgggaatgagtaaaggtgtggttttgactcacaaaaacttcattgcctcttctttaatggtgacaatggagcaagacctagttggagagatggataatgtgtttctatgctttttgccaatgtttcatgtatttggtttggctatcatcacctatgctcagttgcagagaggaaacactgttatttcaatggcgagatttgaccttgagaagatgttaaaagatgtggaaaagtataaagttacccatttgtgggttgtgcctcctgtgatactggctctgagtaagaacagtatggtgaagaagtttaatctttcttctataaagtatattggctccggtgcagctcctttgggcaaagatttaatggaggagtgctctaaggttgttccttatggtattgttgctcagggatatggtatgacagaaacttgtgggattgtatccatggaggatataagaggaggtaaacgaaatagtggttcagctggaatgctggcatctggagtagaagcccagatagttagtgtagatacactgaagcccttacctcctaatcaattgggggagatatgggtgaaggggcctaatatgatgcaaggttacttcaataacccacaggcaaccaagttgactatagataagaaaggttgggtacatactggtgatcttggatattttgatgaagatggacatctttatgttgttgaccgtataaaagagctcatcaaatataaaggatttcaggttgctcctgctgagcttgaaggattgcttgtttctcaccctgaaatactcgatgctgttgtgattccatttcctgacgctgaagcgggtgaagtcccagttgcttatgttgtgcgctctcccaacagttcattaaccgaaaatgatgtgaagaaatttatcgcgggccaggttgcatctttcaaaagattgagaaaagtaacatttataaacagtgtcccgaaatctgcttcggggaaaatcctcagaagagaactcattcagaaagtacgctccaacatgtgatagatactatcattgatataagaaactatctctaaagagatccaactgttaaaaaataaagttagtttagaattttatggtatatttttcattacaaagtatctcagagacgcatactagtttcctaattatatagaagaaagtagcaaacaaagacatggttttagaattttttcttttaaaatttagtgatttgattccttactacaaaaaatcccaaatgttttagaaactattgttgatattgtattataaactatatatgaatggttcctaaataaaactatatgtgaaaaatattgtattata

SEQ ID NO: 3 - DNA, 1158 bp, synthetic construct
  source 1..1158: mol_type = other DNA
atgaatcatcttcgtgctgagggtccggcctccgttctcgccatcggcaccgccaatccggagaacattttaatacaagatgagtttcctgactactactttcgggtcaccaaaagtgaacacatgactcaactcaaagaaaagtttcgaaaaatatgtgacaaaagtatgataaggaaacgtaactgtttcttaaatgaagaacatctaaagcaaaacccaagattggcggagcacgagatgcaaactctggatgcacgtcaagacatgttggtagttgaggttccaaaacttgggaaggatgcttgtgcaaaggccatcaaagaatggggtcaacccaagtctaaaatcactcatttaatcttcactagcgcatcaaccactgacatgcccggtgcagactaccattgcgctaagcttctcggactcagtccctcagtgaagcgtgtgatgatgtatcaactaggctgttatggtggtggaaccgttctacgcattgccaaggacatagcagagaataacaaaggcgcacgagttctcgccgtctgttgtgacataatggcttgcttgtttcgtgggccttcagattctgacctcgaattactagtgggacaagctatctttggtgatggggctgctgctgtcattgttggagccgaacccgatgagtcagttggggaaaggccgatatttgagttagtgtcaactgggcaaacaatcttaccaaactcggaaggaactattgggggacatataagggaagcaggactgatatttgatttacataaggatgtgcctatgttgatctctaataatattgagaaatgtttgattgaggcatttactcctattgggattagtgattggaactctatattttggattacacacccaggtgggaaagctattttggacaaagtggaggagaagttggatctgaagaaggagaagtttgtggattcacgtcatgtgctgagtgagcatgggaatatgtctagctcaactgtcttgtttgttatggatgagttgaggaagaggtcgttggaggaagggaagtctaccactggagatggatttgagtggggtgttctttttgggtttggaccaggtttgactgtcgaaagagtggtcgtgcgtagtgttcccatcaaatattaa

SEQ ID NO: 4 - DNA, 306 bp, synthetic construct
  source 1..306: mol_type = other DNA
atggcagtgaagcatttgattgtattgaagttcaaagatgaaatcacagaagcccaaaaggaagaatttttcaagacgtatgtgaatcttgtgaatatcatcccagccatgaaagatgtatactggggtaaagatgtgactcaaaagaataaggaagaagggtacactcacatagttgaggtaacatttgagagtgtggagactattcaggactacattattcatcctgcccatgttggatttggagatgtctatcgttctttctgggaaaaacttctcatttttgactacacaccacgaaagtag

SEQ ID NO: 5 - DNA, 1364 bp, synthetic construct
  source 1..1364: mol_type = other DNA
tcactatgttattctaaccctttctttccctcattttttcttaatattcaatcaataataatcttcatgggactctcatcagtttgtaccttttcatttcaaactaattaccatactttattaaatcctcacaataataatcccaaaacctcattattatgttatcgacaccccaaaacaccaattaaatactcttacaataattttccctctaaacattgctccaccaagagttttcatctacaaaacaaatgctcagaatcattatcaatcgcaaaaaattccattagggcagctactacaaatcaaactgagcctccagaatctgataatcattcagtagcaactaaaattttaaactttgggaaggcatgttggaaacttcaaagaccatatacaatcatagcatttacttcatgcgcttgtggattgtttgggaaagagttgttgcataacacaaatttaataagttggtctctgatgttcaaggcattcttttttttggtggctgtattatgcattgcttcttttacaactaccatcaatcagatttacgatcttcacattgacagaataaacaagcctgatctaccactagcttcaggggaaatatcagtaaacacagcttggattatgagcataattgtggcactgtttggattgataataactataaaaatgaagggtggaccactctattatttggctactgttttggtatttttggtgggattgtctattctgttccaccatttagatggaagcaaaatccttccactgcatttcttctcaatttcctggcccatattattacaaatttcacattttattatgccagcagagcagctcttggcctaccatttgagttgaggccttcttttactttcctgctagcatttatgaaatcaatgggttcagctttggctttaatcaaagatgcttcagacgttgaaggcgacactaaatttggcatatcaaccttggcaagtaaatatggttccagaaacttgacattattttgttctggaattgttctcctatcctatgtggctgctatacttgctgggattatctggccccaggctttcaacagtaacgtaatgttactttctcatgcaatcttagcattttggttaatcctccagactcgagattttgcgttaacaaattacgacccggaagcaggcagaagattttacgagttcatgtggaagctttattatgctgaatatttagtatatgttttcatataattaagcacattaggtgtccaacacattatattatttatagctaaatattcgtataatttagtggtggatcttacctatttaaatttaaatgtatgcattatttgtttacag

SEQ ID NO: 6 - DNA, 1197 bp, synthetic construct
  source 1..1197: mol_type = other DNA
atgggactctcattagtttgtaccttttcatttcaaactaattatcatactttattaaaccctcataataagaatcccaaaaactcattattatcttatcaacaccccaaaacaccaataattaaatcctcttatgataattttccctctaaatattgcttaaccaagaactttcatttacttggactcaattcacacaacagaataagctcacaatcaaggtccattagggcaggtagcgatcaaattgaaggttctcctcatcatgaatctgataattcaatagcaactaaaattttaaattttggacatacttgttggaaacttcaaagaccatatgtagtaaaagggatgatttcaatcgcttgtggtttgtttgggagagagttgttcaataacagacatttattcagttggggtttgatgtggaaggcattctttgctttggtgcctatattgtccttcaatttctttgcagcaatcatgaatcaaatttacgatgtggacatcgacaggataaacaagcctgatctaccactagtttcaggggaaatgtcaattgaaacagcttggattttgagcataattgtggcactaactgggttgatagtaactataaaattgaaatctgcaccactttttgttttcatttacatttttggtatatttgctgggtttgcctattctgttccaccaattagatggaagcaatatccttttaccaattttctaattaccatatcgagtcatgtgggcttagctttcacatcatattctgcaaccacatcagctcttggtttaccatttgtgtggaggcctgcttttagtttcatcatagcattcatgacagttatgggtatgactattgcttttgccaaagatatttcagatattgaaggcgacgccaaatatggggtatcaactgttgcaaccaaattaggtgctaggaacatgacatttgttgtttctggagttcttcttctaaactacttggtttctatatctattgggataatttggcctcaggttttcaagagtaacataatgatactttctcatgcaatcttagcattttgcttaatcttccagactcgtgagcttgctctagcaaattacgcctcggcgccaagcagacaattcttcgagtttatctggttgctatattatgctgaatactttgtatatgtatttatataa

SEQ ID NO: 7 - DNA, 1638 bp, synthetic construct
  source 1..1638: mol_type = other DNA
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgatgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 8 - DNA, 1635 bp, synthetic construct
  source 1..1635: mol_type = other DNA
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcttccacggcatcgtcattaa

(19) United States
(12) Patent Application Publication
(10) Pub. No.: US 2025/0197797 A1
(43) Pub. Date: Jun. 19, 2025

(54) TRANSGENIC HELICHRYSUM UMBRACULIGERUM CELL, TISSUE, OR PLANT

(71) Applicant: YEDA RESEARCH AND DEVELOPMENT CO. LTD., Rehovot (IL)

(72) Inventors: Asaph AHARONI, Tel Aviv (IL); Prashant SONAWANE, Rehovot (IL); Adam JOZWIAK, Rehovot (IL); Paula BERMAN, Petach Tikva (IL); Luis DE-HARO, Rehovot (IL)

(21) Appl. No.: 18/845,798

(22) PCT Filed: Mar. 9, 2023
(86) PCT No.: PCT/IL2023/050249

§ 371 Date: Sep. 10, 2024

Related U.S. Application Data
(60) Provisional application No. 63/318,849, filed on Mar. 11, 2022.

(51) Int. Cl. C12N 5/04; C07D 311/80

(52) U.S. Cl. CPC C12N 5/04; C07D 311/80; C12N 2510/00

(57) ABSTRACT

The present invention provides a transgenic Helichrysum umbraculigerum (Less.) cell, tissue, or plant, comprising an artificial DNA molecule. Further provided are a method for producing the transgenic H. umbraculigerum (Less.) cell. tissue, or plant, and a method of using same, such as for synthesizing a cannabinoid, a precursor thereof, or both.

### CROSS-REFERENCE TO RELATED APPLICATIONS

This application claims the benefit of priority of U.S. Provisional Patent Application No. 63/318,849, titled “TRANSGENIC HELICHRYSUM UMBRACULIGERUM CELL, TISSUE, OR PLANT”, filed Mar. 11, 2022, the content of which are incorporated herein by reference in their entirety.

### REFERENCE TO AN ELECTRONIC SEQUENCE LISTING

The contents of the electronic sequence listing (YEDA-P-009-PCT.xml; size: 19,755 bytes; and date of creation: Mar. 9, 2023) is herein incorporated by reference in its entirety.

### FIELD OF INVENTION

The present invention relates to a transgenic Helichrysum umbraculigerum cell, tissue, or plant, methods of producing same and use thereof, such for synthesizing a cannabinoid or any precursor thereof.

### BACKGROUND

Cannabinoids are a class of specialized compounds synthesized by Cannabis. They are formed by condensation of terpene and phenol precursors. They include these more abundant forms: Δ⁹-tetrahydrocannabinol (THC), cannabidiol (CBD), cannabichromene (CBC), and cannabigerol (CBG). Another cannabinoid, cannabinol (CBN), is formed from THC as a degradation product and can be detected in some plant strains. Typically, THC, CBD, CBC, and CBG occur together in different ratios in the various plant strains.

Cannabinoids are generally classified into two types, neutral cannabinoids and cannabinoid acids, based on whether they contain a carboxyl group or not. It is known that, in fresh plants, the concentrations of neutral cannabinoids are much lower than those of cannabinoid acids. One strain Cannabis sativa contains approximately 61 compounds belonging to the general class of cannabinoids. These cannabinoids are generally lipophilic, nitrogen-free, mostly phenolic compounds, and are derived biogenetically from a monoterpene and phenol, the acid cannabinoids from a monoterpene and phenol carboxylic acid and have a C21 to base material.

Cannabinoids also find their corresponding carboxylic acids in plant products. In general, the carboxylic acids have the function of a biosynthetic precursor. For example, these compounds arise in vivo from the THC carboxylic acids by decarboxylation the tetrahydrocannabinols Δ⁹- and Δ⁸-THC and CBD from the associated cannabidiol. THC and CBD may be derived artificially from their acidic precursor's tetrahydrocannabinolic acid (THCA) and cannabidiolic acid (CBDA) by non-enzymatic decarboxylation.

Phytocannabinoids, which are plant-based cannabinoids, have also been suggested to be synthesized in plant other than the genus Cannabis.

To this end, Helichrysum umbraculigerum, a flowering plant from Southern Africa was initially suggested to produce cannabinoid-like compounds, such as cannabigerol (CBG) and its precursor cannabigerolic acid (CBGA) in its aerial parts. Nonetheless, a recent publication failed to confirm the presence of these cannabinoids, or others.

### SUMMARY

According to a first aspect, there is provided a transgenic Helichrysum umbraculigerum (Less.) cell, tissue, or plant, comprising an artificial DNA molecule comprising a first nucleic acid sequence selected form the group consisting of: (a) SEQ ID NO: 1 or a functional analog thereof having at least 80% sequence homology to SEQ ID NO: 1; (b) SEQ ID NO: 2 or a functional analog thereof having at least 80% sequence homology to SEQ ID NO: 2; (c) SEQ ID NO: 3 or a functional analog thereof having at least 80% sequence homology to SEQ ID NO: 3; (d) SEQ ID NO: 4 or a functional analog thereof having at least 80% sequence homology to SEQ ID NO: 4; (c) SEQ ID NO: 5 or a functional analog thereof having at least 80% sequence homology to SEQ ID NO: 5; (f) SEQ ID NO: 6 or a functional analog thereof having at least 80% sequence homology to SEQ ID NO: 6; (g) SEQ ID NO: 7 or a functional analog thereof having at least 80% sequence homology to SEQ ID NO: 7; (h) SEQ ID NO: 8 or a functional analog thereof having at least 80% sequence homology to SEQ ID NO: 8; and (i) any combination of (a) to (h).

According to another aspect, there is provided an extract or a fraction thereof, derived from any one of the transgenic H. umbraculigerum cell, tissue, and plant, of the invention, and any combination thereof.

According to another aspect, there is provided a composition comprising the herein disclosed extract or a fraction thereof, and an acceptable carrier.

According to another aspect, there is provided a method for producing the transgenic H. umbraculigerum cell or tissue of the invention, comprising providing a H. umbraculigerum cell or tissue and introducing the artificial DNA molecule comprising the first nucleic acid sequence selected from the group consisting of: SEQ ID Nos: 1-8, a functional analog having at least 80% thereto, and any combination thereof, to the cell or tissue, thereby producing the transgenic H. umbraculigerum cell or tissue.

According to another aspect, there is provided method for producing a cannabinoid or a precursor thereof, comprising: (a) providing a transgenic H. umbraculigerum cell, tissue, or plant, comprising an artificial DNA molecule comprising a first nucleic acid sequence selected from the group consisting of: SEQ ID Nos: 1-8, a functional analog having at least 80% thereto, and any combination thereof; and (b) culturing the transgenic H. umbraculigerum cell, tissue, or plant, such that a cannabinoid or a precursor thereof is produced, thereby producing the cannabinoid or a precursor thereof.

According to another aspect, there is provided a cannabinoid or a precursor thereof, produced according to the herein disclosed method.

According to another aspect, there is provided an extract comprising a cannabinoid or a precursor thereof, obtained according to the herein disclosed method.

According to another aspect, there is provided a composition comprising: (a) the herein disclosed cannabinoid or a precursor thereof; (b) the herein disclosed extract; or (c) a combination of (a) and (b), and an acceptable carrier.

In some embodiments, the artificial DNA molecule comprises a first nucleic acid sequence comprising SEQ ID NO: 3 and SEQ ID NO: 4, or a functional analog thereof having at least 80% sequence homology thereto.

In some embodiments, the artificial DNA molecule further comprises at least one promoter for transcription in a plant cell.

In some embodiments, the at least one promoter is operably linked to any one of SEQ ID Nos: 1-8.

In some embodiments, the at least one promoter is a constitutive promoter or an inducible promoter.

In some embodiments, the artificial DNA molecule further comprises a second nucleic acid sequence encoding at least one protein or enzyme related to cannabinoid synthesis or regulation thereof.

In some embodiments, the extract or a fraction thereof comprises at least one cannabinoid, a precursor thereof, or any combination thereof.

In some embodiments, the method further comprises a step of regenerating the transgenic H. umbraculigerum cell or tissue into a plant, thereby producing a transgenic H. umbraculigerum plant.

In some embodiments, the cell or tissue are obtained or derived from a transformable explant.

In some embodiments, the transformable explant is selected from the group consisting of: a callus, an embryo, and a cell suspension.

In some embodiments, the artificial DNA molecule is a plasmid or an agrobacterium comprising the first nucleic acid sequence.

In some embodiments, the artificial DNA molecule further comprises a second nucleic acid sequence encoding at least one protein or enzyme related to cannabinoid synthesis or regulation thereof.

In some embodiments, any one of the transgenic H. umbraculigerum cell, tissue, and plant, is characterized by being capable of synthesizing at least one cannabinoid, a precursor thereof, or both.

In some embodiments, the method further comprises a step proceeding step (b) comprising extracting the cannabinoid or a precursor thereof from the cultured transgenic H. umbraculigerum cell, tissue, or plant.

In some embodiments, the extracting comprises extracting a tissue derived from the transgenic H. umbraculigerum plant comprising a chlorophyll, a trichome, or both.

In some embodiments, the extracting comprises extracting a stem, a leaf, a portion thereof, or any combination thereof, of the transgenic H. umbraculigerum plant.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B include graphs and an illustration showing in vivo reconstruction of the core cannabinoid pathway in a heterologous system. Co-expression of different combinations of Helichrysum endogenous cannabinoidogenesis related genes, e.g., HuCoAT6, HuTKS4, and HuCBGAS4, along with olivetolic acid cyclase (OAC) and olivetol synthase (OLS) from Cannabis (CsOAC and CsOLS, respectively) in Nicotiana benthamiana leaves. Grey, yellow, and green boxes to the left of the graphs indicate biosynthetic genes that are included in a co-expression experiment; blue boxes mark supplementation of geranyl pyrophosphate (GPP) and either (1A) sodium hexanoate (HexNa) or (1B) OA. Peak areas were used for the comparisons (mean±s.d.; n=3-6). N. benthamiana produced mainly glycosylated products (identified according to in vitro UGT enzyme assays). All the metabolites were identified by exact mass, retention time and MS/MS spectra (See FIG. 2). EV, empty vector.

FIGS. 2A-2C include an illustration, a scheme, chromatograms, and spectra showing reconstruction of the core cannabinoid pathway in a heterologous system. (2A) Schematic representation of products observed in N. benthamiana leaves following co-expression of different combinations of Helichrysum endogenous cannabinoidogenesis related genes, e.g., HuCoAT6, HuTKS4, and HuCBGAS4, along with CsOAC from Cannabis. NbUGT, N. benthamiana uridine diphosphate-glycosyltransferase; HexNa, sodium hexanoate; GPP, geranyl pyrophosphate; OA, olivetolic acid. (2B) Extracted ion chromatograms and MS/MS spectra showing glycosylated OA (Glc-OA), glycosylated polycaprophenone (Glc-PCP1/2) and glycosylated naringenin chalcone (Glc-Naringenin chalcone 1/2) following feeding with HexNa and GPP (I). (2C) Glycosylated cannabigerolic acid (Glc-CBGA) following feeding with OA and GPP (II). Glycosylated metabolites synthesized by the recombinant stevia (SrUGT) or rice (OsUGT) enzymes were used as reference for identification of N. benthamiana products according to exact mass, retention time and MS/MS spectra. EV, empty vector; UDP-Glc, uridine diphosphate glucose.

### DETAILED DESCRIPTION

The present invention, in some embodiments, is directed to transgenic Helichrysum umbraculigerum (Less.) cell, tissue, or plant, comprising an artificial DNA molecule, including methods of using same.

According to one aspect, there is provided a transgenic Helichrysum umbraculigerum (Less.) cell, tissue, or plant, comprising an artificial DNA molecule comprising a first nucleic acid sequence selected form SEQ ID Nos: 1-8, a functional analog(s) thereof having at least 80% sequence homology thereto, or any combination thereof.

According to another aspect, there is provided a transgenic Helichrysum umbraculigerum (Less.) cell, tissue, or plant, comprising an artificial DNA molecule comprising a first nucleic acid sequence comprising SEQ ID NO: 3 or a functional analog thereof having at least 80% sequence homology thereto, and SEQ ID NO: 4 or a functional analog thereof having at least 80% sequence homology thereto.

According to another aspect, there is provided a transgenic Helichrysum umbraculigerum (Less.) cell, tissue, or plant, comprising an artificial DNA molecule comprising a nucleic acid sequence encoding cannabinoidogenesis related genes derived from Cannabis sativa.

In some embodiments, the artificial DNA molecule comprises a nucleic acid sequence encoding olivetol synthase (OLS), olivetolic acid cyclase (OAC), or both of Cannabis sativa, CsOLS, CsOAC, respectively.

In some embodiments, the transgenic Helichrysum umbraculigerum (Less.) cell, tissue, or plant, comprises a nucleic acid sequence encoding CsOLS, CsOAC. In some embodiments, the transgenic Helichrysum umbraculigerum (Less.) cell, tissue, or plant of the invention comprises endogenous nucleic acid sequence(s) encoding cannabinoidogenesis related genes of Helichrysum umbraculigerum (Less.).

In some embodiments, the transgenic Helichrysum umbraculigerum (Less.) cell, tissue, or plant of the invention comprises intact, wild-type, non-modified, endogenous, or any combination thereof, nucleic acid sequence(s) encoding cannabinoidogenesis related genes of Helichrysum umbraculigerum (Less.).

In some embodiments, the intact, wild-type, non-modified, endogenous, or any combination thereof, nucleic acid sequence(s) encoding cannabinoidogenesis related genes of Helichrysum umbraculigerum (Less.) encode: acyl activating enzyme, olivetol synthase, olivetolic acid cyclase, prenyltransferase, tetrahydrocannabinolic acid synthase, cannabidiolic acid synthase, uridine 5′-diphospho-glucuronosyltransferase, alcohol acyl-transferase, or any combination thereof.

In some embodiments, the genome of the transgenic Helichrysum umbraculigerum (Less.) cell, tissue, or plant of the invention, is characterized by or comprises nucleic acid sequence(s) encoding endogenous Helichrysum umbraculigerum (Less.): acyl activating enzyme, olivetol synthase, olivetolic acid cyclase, prenyltransferase, tetrahydrocannabinolic acid synthase, cannabidiolic acid synthase, uridine 5′-diphospho-glucuronosyltransferase, alcohol acyl-transferase, or any combination thereof.

In some embodiments, the artificial DNA molecule comprises a first nucleic acid sequence.

In some embodiments, the first nucleic acid sequence comprises or consists of

(SEQ ID NO: 1)
TTAGATCAAATAACCCGTCCCGAAATCCAATATATATATATATATAATAT
TCAAACTCTCTCTTTCTATCTTCGTACAGTTTAATAGAAGTAATAATGGG
TAAGAATTACAAGTCCCTGGACTCTGTTGTGGCCTCTGACTTCATAGCCC
TAGGTATCACCTCTGAAGTTGCTGAGACACTCCATGGTAGACTGGCCGAG
ATCGTGTGTAATTATGGCGCTGCCACTCCCCAAACATGGATCAATATTGC
CAACCATATTCTGTCGCCTGACCTCCCCTTCTCCCTGCACCAGATGCTCT
TCTATGGTTGCTATAAAGACTTTGGACCTGCCCCTCCTGCTTGGATACCC
GACCCGGAGAAAGTAAAGTCCACCAATCTGGGCGCACTTTTGGAGAAGCG
AGGAAAAGAGTTTTTGGGAGTCAAGTATAAGGATCCCATTTCAAGCTTTT
CTCATTTCCAAGAATTTTCTGTAAGAAACCCTGAGGTGTATTGGAGAACA
GTACTAATGGATGAGATGAAGATAAGTTTTTCAAAGGATCCAGAATGTAT
ATTGCGTAGAGATGATATTAATAATCCAGGGGGTAGTGAATGGCTTCCAG
GAGGTTATCTTAACTCAGCAAAGAATTGCTTGAATGTAAATAGTAACAAG
AAATTGAATGATACAATGATTGTATGGCGTGATGAAGGAAATGATGATTT
GCCTCTAAACAAATTGACACTTGACCAATTGCGTAAACGTGTTTGGTTAG
TTGGTTATGCACTTGAAGAAATGGGTTTGGAGAAGGGTTGTGCAATTGCA
ATTGATATGCCAATGCATGTGGATGCTGTGGTTATCTATCTAGCTATTGT
TCTTGCGGGATATGTAGTTGTTTCTATTGCTGATAGTTTTTCTGCTCCTG
AAATATCAACAAGACTTCGACTATCAAAAGCAAAAGCCATTTTTACACAG
GATCATATTATTCGTGGGAAGAAGCGTATTCCCTTATACAGTAGAGTTGT
GGAAGCCAAGTCTCCCATGGCCATTGTTATTCCTTGTAGTGGCTCTAATA
TTGGTGCAGAATTGCGTGATGGCGATATTTCTTGGGATTACTTTCTAGAA
AGAGCAAAAGAGTTTAAAAATTGTGAATTTACTGCTAGAGAACAACCAGT
TGATGCCTATACAAACATCCTCTTCTCATCTGGAACAACAGGGGAGCCAA
AGGCAATTCCATGGACTCAAGCAACTCCTTTAAAAGCAGCTGCAGATGGG
TGGAGCCATTTGGACATTAGGAAAGGTGATGTCATTGTTTGGCCCACTAA
TCTTGGTTGGATGATGGGTCCTTGGCTGGTCTATGCTTCACTCCTTAATG
GGGCTTCTATTGCCTTGTATAATGGATCACCACTTGTTTCTGGCTTTGCC
AAATTTGTGCAGGATGCTAAAGTAACAATGCTAGGTGTGGTCCCTAGTAT
TGTTCGATCATGGAAAAGTACCAATTGTGTTAGTGGCTATGATTGGTCCA
CCATCCGTTGCTTTTCCTCTTCTGGTGAAGCATCTAATGTAGATGAATAC
CTATGGTTGATGGGGAGAGCAAACTACAAGCCTGTTATCGAAATGTGTGG
TGGCACAGAAATTGGTGGTGCATTTTCTGCTGGCTCTTTCTTACAAGCTC
AATCATTATCTTCATTTAGTTCACAATGTATGGGTTGCACTTTATACATA
CTTGACAAGAATGGTTATCCAATGCCTAAAAACAAACCAGGAATTGGTGA
ATTAGCGCTTGGTCCAGTCATGTTTGGAGCATCGAAGACTCTGTTGAATG
GTAATCACCATGATGTTTATTTTAAGGGAATGCCTACATTGAATGGAGAG
GTTTTAAGGAGGCATGGGGACATTTTTGAGCTTACATCTAATGGTTATTA
TCATGCACATGGTCGTGCAGATGATACAATGAATATTGGAGGCATCAAGA
TTAGTTCCATAGAGATTGAACGAGTTTGTAATGAAGTTGATGACAGAGTT
TTCGAGACAACTGCTATTGGAGTGCCACCTTTGGGCGGTGGACCTGAGCA
ATTAGTAATTTTCTTTGTATTAAAAGATTCAAATGATACAACTATTGACT
TAAATCAATTGAGGTTATCTTTCAACTTGGGTTTACAGAAGAAACTAAAT
CCTCTGTTCAAGGTCACTCGTGTTGTGCCTCTTTCATCACTTCCGAGAAC
AGCAACCAACAAGATCATGAGAAGGGTTTTGCGCCAGCAATTTTCTCACT
TTGAATGAAGTGTTATAATATAATACATACATATATGCTTTGAATAAAGT
GTAGTTGATTACATAGAGCAGTTTGCATTATCAAATGCACTAATAATAAG
TTAAATGCTCTAAAATATGTGTTAAATCTTATGGCAGGATTATAGATTTG
ATTAAGATAGTCATATTGAACCATAAATTTTCATTCAACACATTTTTAGT
GCATTTGACATTATTTTTATTGAATTTAATAAATACTGACTACATTGTAT
AGTCCACTACATCAT.

In some embodiments, the first nucleic acid sequence comprises or consists of

(SEQ ID NO: 2)
CACGAGGGTCACCTACATTCATCCAAATAACAGGTCTCTGTTCTATTCTC
CAATCCAATGGAGAAATCTGGGTATGGAAGAGACGGTATTTACAGGTCTC
TGAGACCACCTCTACACCTCCCCAACAACAACAACCTCTCAATGGTTTCA
TTCCTTTTCAGAAACTCATCTTCATACCCACAAAAGCCAGCTCTCATTGA
TTCCGAAACCAACCAAATACTCTCCTTTTCCCACTTCAAATCTACGGTTA
TCAAGGTCTCCCATGGCTTTCTCAATCTGGGTATCAAGAAAAACGACGTC
GTTCTCATCTACGCCCCTAATTCTATCCACTTCCCTGTTTGTTTCCTGGG
AATTATAGCCTCTGGAGCCATTGCCACTACCTCAAATCCTCTCTACACAG
TTTCCGAGCTTTCCAAACAGGTCAAGGATTCCAATCCCAAACTCATTATC
ACCGTTCCTCAACTCTTGGAAAAAGTAAAGGGTTTCAATCTCCCCACGAT
TCTAATTGGTCCTGATTCTGAACAAGAATCTTCTAGTGATAAAGTAATGA
CCTTTAACGATTTGGTCAACTTAGGTGGGTCGTCTGGCTCAGAATTTCCA
ATTGTTGATGATTTTAAGCAGAGTGACACTGCTGCGCTATTGTACTCATC
TGGCACAACGGGAATGAGTAAAGGTGTGGTTTTGACTCACAAAAACTTCA
TTGCCTCTTCTTTAATGGTGACAATGGAGCAAGACCTAGTTGGAGAGATG
GATAATGTGTTTCTATGCTTTTTGCCAATGTTTCATGTATTTGGTTTGGC
TATCATCACCTATGCTCAGTTGCAGAGAGGAAACACTGTTATTTCAATGG
CGAGATTTGACCTTGAGAAGATGTTAAAAGATGTGGAAAAGTATAAAGTT
ACCCATTTGTGGGTTGTGCCTCCTGTGATACTGGCTCTGAGTAAGAACAG
TATGGTGAAGAAGTTTAATCTTTCTTCTATAAAGTATATTGGCTCCGGTG
CAGCTCCTTTGGGCAAAGATTTAATGGAGGAGTGCTCTAAGGTTGTTCCT
TATGGTATTGTTGCTCAGGGATATGGTATGACAGAAACTTGTGGGATTGT
ATCCATGGAGGATATAAGAGGAGGTAAACGAAATAGTGGTTCAGCTGGAA
TGCTGGCATCTGGAGTAGAAGCCCAGATAGTTAGTGTAGATACACTGAAG
CCCTTACCTCCTAATCAATTGGGGGAGATATGGGTGAAGGGGCCTAATAT
GATGCAAGGTTACTTCAATAACCCACAGGCAACCAAGTTGACTATAGATA
AGAAAGGTTGGGTACATACTGGTGATCTTGGATATTTTGATGAAGATGGA
CATCTTTATGTTGTTGACCGTATAAAAGAGCTCATCAAATATAAAGGATT
TCAGGTTGCTCCTGCTGAGCTTGAAGGATTGCTTGTTTCTCACCCTGAAA
TACTCGATGCTGTTGTGATTCCATTTCCTGACGCTGAAGCGGGTGAAGTC
CCAGTTGCTTATGTTGTGCGCTCTCCCAACAGTTCATTAACCGAAAATGA
TGTGAAGAAATTTATCGCGGGCCAGGTTGCATCTTTCAAAAGATTGAGAA
AAGTAACATTTATAAACAGTGTCCCGAAATCTGCTTCGGGGAAAATCCTC
AGAAGAGAACTCATTCAGAAAGTACGCTCCAACATGTGATAGATACTATC
ATTGATATAAGAAACTATCTCTAAAGAGATCCAACTGTTAAAAAATAAAG
TTAGTTTAGAATTTTATGGTATATTTTTCATTACAAAGTATCTCAGAGAC
GCATACTAGTTTCCTAATTATATAGAAGAAAGTAGCAAACAAAGACATGG
TTTTAGAATTTTTTCTTTTAAAATTTAGTGATTTGATTCCTTACTACAAA
AAATCCCAAATGTTTTAGAAACTATTGTTGATATTGTATTATAAACTATA
TATGAATGGTTCCTAAATAAAACTATATGTGAAAAATATTGTATTATA.

In some embodiments, the first nucleic acid sequence comprises or consists of

(SEQ ID NO: 3)
ATGAATCATCTTCGTGCTGAGGGTCCGGCCTCCGTTCTCGCCATCGGCAC
CGCCAATCCGGAGAACATTTTAATACAAGATGAGTTTCCTGACTACTACT
TTCGGGTCACCAAAAGTGAACACATGACTCAACTCAAAGAAAAGTTTCGA
AAAATATGTGACAAAAGTATGATAAGGAAACGTAACTGTTTCTTAAATGA
AGAACATCTAAAGCAAAACCCAAGATTGGCGGAGCACGAGATGCAAACTC
TGGATGCACGTCAAGACATGTTGGTAGTTGAGGTTCCAAAACTTGGGAAG
GATGCTTGTGCAAAGGCCATCAAAGAATGGGGTCAACCCAAGTCTAAAAT
CACTCATTTAATCTTCACTAGCGCATCAACCACTGACATGCCCGGTGCAG
ACTACCATTGCGCTAAGCTTCTCGGACTCAGTCCCTCAGTGAAGCGTGTG
ATGATGTATCAACTAGGCTGTTATGGTGGTGGAACCGTTCTACGCATTGC
CAAGGACATAGCAGAGAATAACAAAGGCGCACGAGTTCTCGCCGTCTGTT
GTGACATAATGGCTTGCTTGTTTCGTGGGCCTTCAGATTCTGACCTCGAA
TTACTAGTGGGACAAGCTATCTTTGGTGATGGGGCTGCTGCTGTCATTGT
TGGAGCCGAACCCGATGAGTCAGTTGGGGAAAGGCCGATATTTGAGTTAG
TGTCAACTGGGCAAACAATCTTACCAAACTCGGAAGGAACTATTGGGGGA
CATATAAGGGAAGCAGGACTGATATTTGATTTACATAAGGATGTGCCTAT
GTTGATCTCTAATAATATTGAGAAATGTTTGATTGAGGCATTTACTCCTA
TTGGGATTAGTGATTGGAACTCTATATTTTGGATTACACACCCAGGTGGG
AAAGCTATTTTGGACAAAGTGGAGGAGAAGTTGGATCTGAAGAAGGAGAA
GTTTGTGGATTCACGTCATGTGCTGAGTGAGCATGGGAATATGTCTAGCT
CAACTGTCTTGTTTGTTATGGATGAGTTGAGGAAGAGGTCGTTGGAGGAA
GGGAAGTCTACCACTGGAGATGGATTTGAGTGGGGTGTTCTTTTTGGGTT
TGGACCAGGTTTGACTGTCGAAAGAGTGGTCGTGCGTAGTGTTCCCATCA
AATATTAA.

In some embodiments, the first nucleic acid sequence comprises or consists of

(SEQ ID NO: 4)
ATGGCAGTGAAGCATTTGATTGTATTGAAGTTCAAAGATGAAATCACAGA
AGCCCAAAAGGAAGAATTTTTCAAGACGTATGTGAATCTTGTGAATATCA
TCCCAGCCATGAAAGATGTATACTGGGGTAAAGATGTGACTCAAAAGAAT
AAGGAAGAAGGGTACACTCACATAGTTGAGGTAACATTTGAGAGTGTGGA
GACTATTCAGGACTACATTATTCATCCTGCCCATGTTGGATTTGGAGATG
TCTATCGTTCTTTCTGGGAAAAACTTCTCATTTTTGACTACACACCACGA
AAGTAG.

In some embodiments, the first nucleic acid sequence comprises or consists of

(SEQ ID NO: 5)
TCACTATGTTATTCTAACCCTTTCTTTCCCTCATTTTTTCTTAATATTCA
ATCAATAATAATCTTCATGGGACTCTCATCAGTTTGTACCTTTTCATTTC
AAACTAATTACCATACTTTATTAAATCCTCACAATAATAATCCCAAAACC
TCATTATTATGTTATCGACACCCCAAAACACCAATTAAATACTCTTACAA
TAATTTTCCCTCTAAACATTGCTCCACCAAGAGTTTTCATCTACAAAACA
AATGCTCAGAATCATTATCAATCGCAAAAAATTCCATTAGGGCAGCTACT
ACAAATCAAACTGAGCCTCCAGAATCTGATAATCATTCAGTAGCAACTAA
AATTTTAAACTTTGGGAAGGCATGTTGGAAACTTCAAAGACCATATACAA
TCATAGCATTTACTTCATGCGCTTGTGGATTGTTTGGGAAAGAGTTGTTG
CATAACACAAATTTAATAAGTTGGTCTCTGATGTTCAAGGCATTCTTTTT
TTTGGTGGCTGTATTATGCATTGCTTCTTTTACAACTACCATCAATCAGA
TTTACGATCTTCACATTGACAGAATAAACAAGCCTGATCTACCACTAGCT
TCAGGGGAAATATCAGTAAACACAGCTTGGATTATGAGCATAATTGTGGC
ACTGTTTGGATTGATAATAACTATAAAAATGAAGGGTGGACCACTCTATT
ATTTGGCTACTGTTTTGGTATTTTTGGTGGGATTGTCTATTCTGTTCCAC
CATTTAGATGGAAGCAAAATCCTTCCACTGCATTTCTTCTCAATTTCCTG
GCCCATATTATTACAAATTTCACATTTTATTATGCCAGCAGAGCAGCTCT
TGGCCTACCATTTGAGTTGAGGCCTTCTTTTACTTTCCTGCTAGCATTTA
TGAAATCAATGGGTTCAGCTTTGGCTTTAATCAAAGATGCTTCAGACGTT
GAAGGCGACACTAAATTTGGCATATCAACCTTGGCAAGTAAATATGGTTC
CAGAAACTTGACATTATTTTGTTCTGGAATTGTTCTCCTATCCTATGTGG
CTGCTATACTTGCTGGGATTATCTGGCCCCAGGCTTTCAACAGTAACGTA
ATGTTACTTTCTCATGCAATCTTAGCATTTTGGTTAATCCTCCAGACTCG
AGATTTTGCGTTAACAAATTACGACCCGGAAGCAGGCAGAAGATTTTACG
AGTTCATGTGGAAGCTTTATTATGCTGAATATTTAGTATATGTTTTCATA
TAATTAAGCACATTAGGTGTCCAACACATTATATTATTTATAGCTAAATA
TTCGTATAATTTAGTGGTGGATCTTACCTATTTAAATTTAAATGTATGCA
TTATTTGTTTACAG.

In some embodiments, the first nucleic acid sequence comprises or consists of

(SEQ ID NO: 6)
ATGGGACTCTCATTAGTTTGTACCTTTTCATTTCAAACTAATTATCATAC
TTTATTAAACCCTCATAATAAGAATCCCAAAAACTCATTATTATCTTATC
AACACCCCAAAACACCAATAATTAAATCCTCTTATGATAATTTTCCCTCT
AAATATTGCTTAACCAAGAACTTTCATTTACTTGGACTCAATTCACACAA
CAGAATAAGCTCACAATCAAGGTCCATTAGGGCAGGTAGCGATCAAATTG
AAGGTTCTCCTCATCATGAATCTGATAATTCAATAGCAACTAAAATTTTA
AATTTTGGACATACTTGTTGGAAACTTCAAAGACCATATGTAGTAAAAGG
GATGATTTCAATCGCTTGTGGTTTGTTTGGGAGAGAGTTGTTCAATAACA
GACATTTATTCAGTTGGGGTTTGATGTGGAAGGCATTCTTTGCTTTGGTG
CCTATATTGTCCTTCAATTTCTTTGCAGCAATCATGAATCAAATTTACGA
TGTGGACATCGACAGGATAAACAAGCCTGATCTACCACTAGTTTCAGGGG
AAATGTCAATTGAAACAGCTTGGATTTTGAGCATAATTGTGGCACTAACT
GGGTTGATAGTAACTATAAAATTGAAATCTGCACCACTTTTTGTTTTCAT
TTACATTTTTGGTATATTTGCTGGGTTTGCCTATTCTGTTCCACCAATTA
GATGGAAGCAATATCCTTTTACCAATTTTCTAATTACCATATCGAGTCAT
GTGGGCTTAGCTTTCACATCATATTCTGCAACCACATCAGCTCTTGGTTT
ACCATTTGTGTGGAGGCCTGCTTTTAGTTTCATCATAGCATTCATGACAG
TTATGGGTATGACTATTGCTTTTGCCAAAGATATTTCAGATATTGAAGGC
GACGCCAAATATGGGGTATCAACTGTTGCAACCAAATTAGGTGCTAGGAA
CATGACATTTGTTGTTTCTGGAGTTCTTCTTCTAAACTACTTGGTTTCTA
TATCTATTGGGATAATTTGGCCTCAGGTTTTCAAGAGTAACATAATGATA
CTTTCTCATGCAATCTTAGCATTTTGCTTAATCTTCCAGACTCGTGAGCT
TGCTCTAGCAAATTACGCCTCGGCGCCAAGCAGACAATTCTTCGAGTTTA
TCTGGTTGCTATATTATGCTGAATACTTTGTATATGTATTTATATAA.

In some embodiments, the first nucleic acid sequence comprises or consists of

(SEQ ID NO: 7)
ATGAATTGCTCAGCATTTTCCTTTTGGTTTGTTTGCAAAATAATATTTTT
CTTTCTCTCATTCCATATCCAAATTTCAATAGCTAATCCTCGAGAAAACT
TCCTTAAATGCTTCTCAAAACATATTCCCAACAATGTAGCAAATCCAAAA
CTCGTATACACTCAACACGACCAATTGTATATGTCTATCCTGAATTCGAC
AATACAAAATCTTAGATTCATCTCTGATACAACCCCAAAACCACTCGTTA
TTGTCACTCCTTCAAATAACTCCCATATCCAAGCAACTATTTTATGCTCT
AAGAAAGTTGGCTTGCAGATTCGAACTCGAAGCGGTGGCCATGATGCTGA
GGGTATGTCCTACATATCTCAAGTCCCATTTGTTGTAGTAGACTTGAGAA
ACATGCATTCGATCAAAATAGATGTTCATAGCCAAACTGCGTGGGTTGAA
GCCGGAGCTACCCTTGGAGAAGTTTATTATTGGATCAATGAGAAGAATGA
GAATCTTAGTTTTCCTGGTGGGTATTGCCCTACTGTTGGCGTAGGTGGAC
ACTTTAGTGGAGGAGGCTATGGAGCATTGATGCGAAATTATGGCCTTGCG
GCTGATAATATTATTGATGCACACTTAGTCAATGTTGATGGAAAAGTTCT
AGATCGAAAATCCATGGGAGAAGATCTGTTTTGGGCTATACGTGGTGGTC
CCATCAAAGTCTACTATATTCAGTGTTAAAAAGAACATGGAGATACATGG
GCTTGTCAAGTTATTTAACAAATGGCAAAATATTGCTTACAAGTATGACA
AAGATTTAGTACTCATGACTCACTTCATAACAAAGAATATTACAGATAAT
CATGGGAAGAATAAGACTACAGTACATGGTTACTTCTCTTCAATTTTTCA
TGGTGGAGTGGATAGTCTAGTCGACTTGATGAACAAGAGCTTTCCTGAGT
TGGGTATTAAAAAAACTGATTGCAAAGAATTTAGCTGGATTGATACAACC
ATCTTCTACAGTGGTGTTGTAAATTTTAACACTGCTAATTTTAAAAAGGA
AATTTTGCTTGATAGATCAGCTGGGAAGAAGACGGCTTTCTCAATTAAGT
TAGACTATGTTAAGAAACCAATTCCAGAAACTGCAATGGTCAAAATTTTG
GAAAAATTATATGAAGAAGATGTAGGAGCTGGGATGTATGTGTTGTACCC
TTACGGTGGTATAATGGAGGAGATTTCAGAATCAGCAATTCCATTCCCTC
ATCGAGCTGGAATAATGTATGAACTTTGGTACACTGCTTCCTGGGAGAAG
CAAGAAGATAATGAAAAGCATATAAACTGGGTTCGAAGTGTTTATAATTT
TACGACTCCTTATGTGTCCCAAAATCCAAGATTGGCGTATCTCAATTATA
GGGACCTTGATTTAGGAAAAACTAATCATGCGAGTCCTAATAATTACACA
CAAGCACGTATTTGGGGTGAAAAGTATTTTGGTAAAAATTTTAACAGGTT
AGTTAAGGTGAAAACTAAAGTTGATCCCAATAATTTTTTTAGAAACGAAC
AAAGTATCCCACCTCTTCCACCGCATCATCATTAA.

In some embodiments, the first nucleic acid sequence comprises or consists of

(SEQ ID NO: 8)
ATGAAGTGCTCAACATTCTCCTTTTGGTTTGTTTGCAAGATAATATTTTT
CTTTTTCTCATTCAATATCCAAACTTCCATTGCTAATCCTCGAGAAAACT
TCCTTAAATGCTTCTCGCAATATATTCCCAATAATGCAACAAATCTAAAA
CTCGTATACACTCAAAACAACCCATTGTATATGTCTGTCCTAAATTCGAC
AATACACAATCTTAGATTCACCTCTGACACAACCCCAAAACCACTTGTTA
TCGTCACTCCTTCACATGTCTCTCATATCCAAGGCACTATTCTATGCTCC
AAGAAAGTTGGCTTGCAGATTCGAACTCGAAGTGGTGGTCATGATTCTGA
GGGCATGTCCTACATATCTCAAGTCCCATTTGTTATAGTAGACTTGAGAA
ACATGCGTTCAATCAAAATAGATGTTCATAGCCAAACTGCATGGGTTGAA
GCCGGAGCTACCCTTGGAGAAGTTTATTATTGGGTTAATGAGAAAAATGA
GAATCTTAGTTTGGCGGCTGGGTATTGCCCTACTGTTTGCGCAGGTGGAC
ACTTTGGTGGAGGAGGCTATGGACCATTGATGAGAAACTATGGCCTCGCG
GCTGATAATATCATTGATGCACACTTAGTCAACGTTCATGGAAAAGTGCT
AGATCGAAAATCTATGGGGGAAGATCTCTTTTGGGCTTTACGTGGTGGTG
GAGCAGAAAGCTTCGGAATCATTGTAGCATGGAAAATTAGACTGGTTGCT
GTCCCAAAGTCTACTATGTTTAGTGTTAAAAAGATCATGGAGATACATGA
GCTTGTCAAGTTAGTTAACAAATGGCAAAATATTGCTTACAAGTATGACA
AAGATTTATTACTCATGACTCACTTCATAACTAGGAACATTACAGATAAT
CAAGGGAAGAATAAGACAGCAATACACACTTACTTCTCTTCAGTTTTCCT
TGGTGGAGTGGATAGTCTAGTCGACTTGATGAACAAGAGTTTTCCTGAGT
TGGGTATTAAAAAAACGGATTGCAGACAATTGAGCTGGATTGATACTATC
ATCTTCTATAGTGGTGTTGTAAATTACGACACTGATAATTTTAACAAGGA
AATTTTGCTTGATAGATCCGCTGGGCAGAACGGTGCTTTCAAGATTAAGT
TAGACTACGTTAAGAAACCAATTCCAGAATCTGTATTTGTCCAAATTTTG
GAAAAATTATATGAAGAAGATATAGGAGCTGGGATGTATGCGTTGTACCC
TTACGGTGGTATAATGGATGAGATTTCAGAATCAGCAATTCCATTCCCTC
ATCGAGCTGGAATCTTGTATGAGTTATGGTACATATGTAGTTGGGAGAAG
CAAGAAGATAACGAAAAGCATCTAAACTGGATTAGAAATATTTATAACTT
CATGACTCCTTATGTGTCCAAAAATCCAAGATTGGCATATCTCAATTATA
GAGACCTTGATATAGGAATAAATGATCCCAAGAATCCAAATAATTACACA
CAAGCACGTATTTGGGGTGAGAAGTATTTTGGTAAAAATTTTGACAGGCT
AGTAAAAGTGAAAACCCTGGTTGATCCCAATAACTTTTTTAGAAACGAAC
AAAGCATCCCACCTCTTCCACGGCATCGTCATTAA.

In some embodiments, the first nucleic acid sequence is selected from: (a) SEQ ID NO: 1 or a functional analog thereof having at least 80% sequence homology to SEQ ID NO: 1; (b) SEQ ID NO: 2 or a functional analog thereof having at least 80% sequence homology to SEQ ID NO: 2; (c) SEQ ID NO: 3 or a functional analog thereof having at least 80% sequence homology to SEQ ID NO: 3; (d) SEQ ID NO: 4 or a functional analog thereof having at least 80% sequence homology to SEQ ID NO: 4; (e) SEQ ID NO: 5 or a functional analog thereof having at least 80% sequence homology to SEQ ID NO: 5; (f) SEQ ID NO: 6 or a functional analog thereof having at least 80% sequence homology to SEQ ID NO: 6; (g) SEQ ID NO: 7 or a functional analog thereof having at least 80% sequence homology to SEQ ID NO: 7; (h) SEQ ID NO: 8 or a functional analog thereof having at least 80% sequence homology to SEQ ID NO: 8; and (i) any combination of (a) to (h).

In some embodiments, the first nucleic acid comprises a combination of SEQ ID Nos: 3 and 4. In some embodiments, the first nucleic acid comprises SEQ ID Nos: 3 and 4.

In some embodiments, at least 80% sequence homology comprises at least 85%, at least 90%, at least 92%, at least 95%, at least 97%, at least 99%, or 100% sequence homology, or any value and range therebetween. Each possibility represents a separate embodiment of the invention. In some embodiments, at least 80% sequence homology comprises 80-90%, 80-95%, 80-99%, 90-97%, 87-97%, or 80-100% sequence homology. Each possibility represents a separate embodiment of the invention.

In some embodiments, the artificial DNA molecule is an isolated polynucleotide. In some embodiments, the artificial DNA molecule is an isolated DNA molecule. In some embodiments, the artificial DNA molecule is a complementary DNA (cDNA) molecule.

In some embodiments, the artificial DNA molecule comprises nucleic acid sequences derived from a Cannabis plant. In some embodiments, the Cannabis plant is or comprises Cannabis sativa. In some embodiments, the artificial DNA molecule comprises nucleic acid sequence derived from plant and involved in the synthesis or production of a cannabinoid or any precursor thereof. In some embodiments, the artificial DNA molecule comprises nucleic acid sequence derived from C. sativa and involved in the synthesis or production of a cannabinoid or any precursor thereof.

In some embodiments, the first nucleic acid sequence comprises or consists of a sequence, as specific in Table 1, hereinbelow.

TABLE 1
Genes involved in the synthesis of a
cannabinoid, or a precursor thereof.

| C. sativa | | Accession | SEQ |
|---|---|---|---|
| genes | Full name | No. | ID NO: |
| AAE1 | Acyl activating enzyme 1 | JN717233 | 1 |
| AAE3 | Acyl activating enzyme 3 | JN717235 | 2 |
| OLS | Olivetol Synthase | AB164375 | 3 |
| OAC | Olivetolic acid cyclase | JN679224 | 4 |
| PT1/GOT1 | Prenyltransferase 1/ | BK010678 | 5 |
| | geranylpyrophosphate:olivetolate | | |
| | geranyltransferase 1 | | |
| PT4/GOT4 | Prenyltransferase 4/ | BK010648 | 6 |
| | geranylpyrophosphate:olivetolate | | |
| | geranyltransferase 4 | | |
| THCAS | Tetrahydrocannabinolic acid synthase | AB057805 | 7 |
| CBDAS | Cannabidiolic acid synthase | AB292682 | 8 |

As used herein, the terms “isolated polynucleotide” and “isolated DNA molecule” refers to a nucleic acid molecule that is essentially free from contaminating cellular components, such as carbohydrate, lipid, or other proteinaceous impurities associated with the nucleic acid in nature. Typically, a preparation of isolated DNA or RNA contains the nucleic acid in a highly purified form, e.g., at least about 80% pure, at least about 90% pure, at least about 95% pure, greater than 95% pure, or greater than 99% pure. In some embodiments, the isolated polynucleotide is any one of DNA, RNA, and cDNA. In some embodiments, the isolated polynucleotide is a synthesized polynucleotide. Synthesis of polynucleotides is well known in the art and may be performed, for example, by ligating or covalently linking by primer linkers multiple nucleic acid molecules together.

The term “nucleic acid” is well known in the art. A “nucleic acid” as used herein will generally refer to any molecule (e.g., a strand) of DNA, RNA or a derivative or analog thereof, comprising nucleotides. Nucleotides are comprised of nucleosides and phosphate groups. The nitrogenous bases of nucleosides include, for example, naturally occurring purine or pyrimidine nucleosides as found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNAs, circular nucleic acids, fragments of genomic DNA or RNA, degraded nucleic acids, amplification products, modified nucleic acids, plasmid or organellar nucleic acids, and artificial nucleic acids such as oligonucleotides.

In some embodiments, the artificial DNA molecule is introduced into the transgenic H. umbraculigerum cell, tissue, or plant using a vector or a plasmid comprising the artificial DNA molecule. comprises a plasmid. In some embodiments, the vector comprises or is an agrobacterium comprising the artificial DNA molecule. In some embodiments, the vector is an expression vector. In some embodiments, the vector is a plant expression vector. In some embodiments, the vector is an artificial vector.

Expressing of a polynucleotide within a cell is well known to one skilled in the art. It can be carried out by, among many methods, transfection, viral infection, or direct alteration of the cell's genome. In some embodiments, the polynucleotide is in an expression vector such as plasmid or viral vector. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the polynucleotide as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation, and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothioncin promoter, murine mammary tumor virus promoter, Rous sarcoma virus promoter, polyhedrin promoter, or other promoters shown effective for expression in eukaryotic cells.

Other types of plant promoters (either constitutive or inducible) are common and would be apparent to one of ordinary skill in the art. Such promoters are referred to, for example, in U.S. Pat. No. 8,395,024, International Patent Application No. PCT/GB 1992/000627, U.S. Pat. Nos. 5,981,727, 6,693,227, which are incorporated herein by reference in their entirety, among others.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a virus.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the artificial vector comprises a polynucleotide as disclosed herein, encoding a protein.

In some embodiments, the artificial DNA molecule further comprises at least one promoter.

In some embodiments, the promoter is suitable for transcription in a plant cell.

In some embodiments, the at least one promoter is operably linked to SEQ ID Nos: 1-8, or any combination thereof.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, the at least one promoter is a constitutive promoter or an inducible promoter.

The terms “inducible” and “controllable” are interchangeable.

Types of promoters, including their sequences, and methods of integration and/or use, are common and would be apparent to one of ordinary skill in the art of molecular biology.

In some embodiments, the promoter is a H. umbraculigerum endogenous promoter.

In some embodiments, the promoter is an exogenous promoter. In some embodiments, an exogenous promoter comprises a viral promoter. In some embodiments, the viral promoter is derived from a plant virus.

In some embodiments, the promoter is a modified promoter.

In some embodiments, a modified promoter is a promoter is characterized by having 99% identity at most to a control promoter (e.g., the unmodified promoter, as described herein). In some embodiments, a gene or a polynucleotide operably linked the modified promoter is more expressed by at least: 5%, at least 10%, 20%, 50%, 100%, 200%, 350%, 500%, 750%, or 1,000%, or any value and range therebetween, compared to the gene or the polynucleotide operably linked a control promoter. Each possibility represents a separate embodiment of the invention.

According to some embodiments, the artificial DNA molecule further comprises a second nucleic acid sequence.

In some embodiments, the second nucleic acid sequence is encoding at least one protein or enzyme related to cannabinoid synthesis or regulation thereof.

In some embodiments, the second nucleic acid sequence is a polynucleotide capable of or configured to regulate the expression of a protein or an enzyme related to cannabinoid synthesis or regulation thereof.

In some embodiments, the polynucleotide capable of or configured to regulate the expression of a protein or an enzyme is characterized by having a nucleic acid sequence having at least 70%, at least 80%, at least 90%, at least 95%, at least 97%, at least 99% or 100% identity or homology to a nucleic acid sequence encoding a protein or an enzyme related to cannabinoid synthesis or regulation thereof, as described herein.

In some embodiments, “being capable of” or “configured to” regulate the expression comprises being complementary to a nucleic acid sequence encoding a protein or an enzyme related to cannabinoid synthesis or regulation thereof, as described herein.

In some embodiments, the polynucleotide capable of or configured to regulate the expression of a protein or an enzyme related to cannabinoid synthesis or regulation thereof, as described herein is an inhibitory nucleic acid.

In some embodiments, the inhibitory nucleic acid is an interfering RNA. In some embodiments, the interfering RNA is a small hairpin RNA (shRNA) or small interfering RNA (siRNA).

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

As used herein “an interfering RNA” refers to any double stranded or single stranded RNA sequence, capable—either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

As used herein “an shRNA” (small hairpin RNA) refers to an RNA molecule comprising an antisense region, a loop portion, and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family.

A “small interfering RNA” or “siRNA” as used herein refers to any small RNA molecule capable of inhibiting or down regulating gene expression by mediating RNA interference in a sequence specific manner. The small RNA can be, for example, about 18 to 21 nucleotides long.

The terms “homology” or “identity”, as used interchangeably herein, refer to sequence identity between two nucleic acid sequences, with identity being a stricter comparison. The phrases “percent identity or homology” and “% identity or homology” refer to the percentage of sequence identity found in a comparison of two nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base then the molecules are identical at that position. A degree of identity between nucleic acid sequences is a function of the number of identical or matching nucleotides at positions shared by the nucleic acid sequences.

The following is a non-limiting example for calculating homology or sequence identity between two sequences (the terms are used interchangeably herein). The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences.

In some embodiments, % homology or identity as described herein are calculated or determined using the basic local alignment search tool (BLAST). In some embodiments, % homology or identity as described herein are calculated or determined using Blossum 62 scoring matrix.

According to some embodiments, there is provided a transgenic H. umbraculigerum cell, tissue, or plant comprising the artificial DNA molecule disclosed herein.

As used herein, the term “transgenic cell” refers to any cell that has undergone human manipulation on the genomic or gene level. In some embodiments, the transgenic cell has had exogenous polynucleotide, such as an artificial DNA molecule as disclosed herein, introduced into it. In some embodiments, a transgenic cell comprises a cell that has an artificial vector introduced into it. In some embodiments, a transgenic cell is a cell which has undergone genome mutation or modification. In some embodiments, a transgenic cell is a cell that has undergone CRISPR genome editing. In some embodiments, a transgenic cell is a cell that has undergone targeted mutation of at least one base pair of its genome. In some embodiments, the exogenous polynucleotide (e.g., the artificial DNA molecule disclosed herein) or vector is stably integrated into the cell. In some embodiments, the transgenic cell expresses an artificial DNA molecule of the invention. In some embodiments, the transgenic cell expresses a vector comprising an artificial DNA molecule of the invention. In some embodiments, the transgenic cell expresses a protein encoded by the artificial DNA molecule of the invention, or a vector comprising same. In some embodiments, the transgenic cell, is a cell that was devoid of an artificial DNA molecule of the invention that has been transformed or genetically modified to include the artificial DNA molecule of the invention. In some embodiments, CRISPR technology is used to modify the genome of the cell, as described herein.

In some embodiments, the cell is a H. umbraculigerum cell or a H. umbraculigerum cell in a culture.

In some embodiments, the tissue is a green tissue. In some embodiments, the tissue comprises a chlorophyll, a trichome, or both.

In some embodiments, the transgenic plant comprises any plant part derived therefrom.

According to some embodiments, the transgenic plant, or any portion, seed, tissue, or organ thereof, comprises at least one transgenic plant cell of the invention.

In some embodiments, the transgenic plant, transgenic plant tissue, or plant part consists of transgenic plant cells of the invention. In some embodiments, the transgenic plant, transgenic plant tissue, or plant part comprises at least: 20%, 25%, 30%, 35%, 40%, 45%, 50%, 55%, 60%, 65%, 70%, 75%, 80%, 85%, 90%, 95%, 97%, or 99% transgenic cells of the invention, or any value and range therebetween. Each possibility represents a separate embodiment of the invention. In some embodiments, the transgenic plant, transgenic plant tissue, or plant part comprises 20%-50%, 20%-60%, 20%-70%, 20%-80%, 20%-90%, or 20%-100% transgenic cells of the invention. Each possibility represents a separate embodiment of the invention.

In some embodiments, the plant part comprises: a leaf, a trichome, a stem, a flower, a root, a seed, any homogenate thereof, any extract thereof, any portion thereof, or any combination thereof.

In some embodiments, a portion comprises a fraction or a plurality thereof.

According to some embodiments, there is provided a homogenate, a lysate, or an extract derived from a transgenic cell disclosed herein, any combination thereof, or any fraction thereof.

According to some embodiments, there is provided an extract derived from a transgenic cell disclosed herein, or any fraction thereof.

In some embodiments, the extract comprises at least one cannabinoid, a precursor thereof, or any combination thereof.

Methods and/or means for extracting, lysing, homogenizing, fractionating, or any combination thereof, a cell or a culture of same, are common and would be apparent to one of ordinary skill in the art of cell biology and biochemistry. Non-limiting examples include, but are not limited to, pressure lysis (e.g., such as using a French press), enzymatic lysis, soluble-insoluble phase separation (such for obtaining a supernatant and a pellet), detergent-based lysis, solvent (e.g., polar, or nonpolar solvent), liquid chromatography mass spectrometry, or others.

According to some embodiments, there is provided a composition comprising the herein disclosed extract or a fraction thereof, and an acceptable carrier.

As used herein, the term “carrier”, “excipient”, or “adjuvant” refers to any component of a composition, e.g., pharmaceutical or nutraceutical, that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Methods of Production

According to some embodiments, there is provided a method for producing the transgenic H. umbraculigerum cell or tissue of the invention.

In some embodiments, the method comprises providing a H. umbraculigerum cell or tissue and introducing an artificial DNA molecule comprising a first nucleic acid sequence selected from: SEQ ID Nos: 1-8, or functional analog having at least 80% thereto, and any combination thereof, to the cell or tissue, thereby producing the transgenic H. umbraculigerum cell or tissue.

In some embodiments, the method further comprises a step of regenerating the transgenic H. umbraculigerum cell or tissue into a plant, thereby producing a transgenic H. umbraculigerum plant.

In some embodiments, the cell or tissue are obtained or derived from a transformable explant.

In some embodiments, the transformable explant is selected from: a callus, an embryo, and a cell suspension.

Methods for obtaining transformable plant explant, and types of same, are common and would be apparent to one of ordinary skill in the art of plant biology and physiology.

In some embodiments, the transgenic H. umbraculigerum cell, tissue, and plant, as disclosed herein, produced according to the herein disclosed method, or both, is characterized by being capable of synthesizing at least one cannabinoid, a precursor thereof, or both.

According to some embodiments, there is provided a method for producing a cannabinoid or a precursor thereof, or any salt thereof.

According to some embodiments, there is provided a method for producing an acyl phloroglucinoid or a precursor thereof, or any salt thereof.

According to some embodiments, there is provided a method for producing an amorfrutin (cannabinoid-like) or a precursor thereof, or any salt thereof.

According to some embodiments, there is provided a method for producing a prenyl chalcone a precursor thereof, or any salt thereof.

In some embodiments, the method comprises: providing and culturing a transgenic H. umbraculigerum cell, tissue, or plant, as disclosed herein, such that a cannabinoid or a precursor thereof is produced.

In some embodiments, the method comprises: (a) providing a transgenic H. umbraculigerum cell, tissue, or plant, comprising an artificial DNA molecule comprising a first nucleic acid sequence selected from: SEQ ID Nos: 1-8, a functional analog having at least 80% homology thereto, or any combination thereof; and (b) culturing the transgenic H. umbraculigerum cell, tissue, or plant, such that a cannabinoid or a precursor thereof is produced.

In some embodiments, the transgenic H. umbraculigerum cell, tissue, or plant, comprises an artificial DNA molecule comprising a first nucleic acid sequence comprising SEQ ID NO: 3 and SEQ ID NO: 4, or functional analog(s) having at least 80% homology thereto.

In some embodiments, the method further comprises a step proceeding the culturing step comprising extracting the cannabinoid or a precursor thereof from the cultured transgenic H. umbraculigerum cell, tissue, or plant.

In some embodiments, the method further comprises a step proceeding step (b) comprising extracting the cannabinoid or a precursor thereof from the cultured transgenic H. umbraculigerum cell, tissue, or plant.

In some embodiments, the method further comprises a step proceeding step (b) comprising extracting the acyl phloroglucinoid, amorfrutin (cannabinoid-like), prenyl chalcone, any precursor thereof, salt thereof, or any combination thereof, from the cultured transgenic H. umbraculigerum cell, tissue, or plant.

In some embodiments, the extracting comprises extracting a tissue derived from the transgenic H. umbraculigerum plant.

In some embodiments, the extracted tissue comprises a chlorophyll, a trichome, or both.

In some embodiments, the extracting comprises extracting a stem, a leaf, a trichome, a portion thereof, or any combination thereof, of the transgenic H. umbraculigerum plant or a tissue derived therefrom.

According to some embodiments, there is provided a cannabinoid or a precursor thereof, produced according to the herein disclosed method.

According to some embodiments, there is provided an acyl phloroglucinoid, an amorfrutin (cannabinoid-like), a prenyl chalcone, any precursor thereof, salt thereof, or any combination thereof, produced according to the herein disclosed method.

According to some embodiments, there is provided an extract comprising a cannabinoid or a precursor thereof, obtained according to the herein disclosed method.

According to some embodiments, there is provided an extract comprising an acyl phloroglucinoid, an amorfrutin (cannabinoid-like), a prenyl chalcone, any precursor thereof, salt thereof, or any combination thereof, obtained according to the herein disclosed method.

According to some embodiments, there is provided a composition comprising: (a) a an acyl phloroglucinoid, an amorfrutin (cannabinoid-like), a prenyl chalcone, any precursor thereof, or any combination thereof, produced according to the herein disclosed method; (b) an extract, produced according to the herein disclosed method; or (c) a combination of (a) and (b), and an acceptable carrier, as disclosed herein.

According to some embodiments, there is provided a composition comprising: (a) a cannabinoid or a precursor thereof, produced according to the herein disclosed method; (b) an extract, produced according to the herein disclosed method; or (c) a combination of (a) and (b), and an acceptable carrier, as disclosed herein.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

### Materials

Unless otherwise stated, all the analytical metabolites were ≥95% pure. CBGA, CBCA, CBDA, acetic acid, propionic acid, butyric acid, pentanoic acid, hexanoic acid, heptanoic acid, octanoic acid, ±2-methyl butyric acid, phenylalanine, hexanoic-D₁₁ acid (D>98%), GPP, IPP, FPP, phloretin, naringenin, malonyl-CoA (≥90%), acetyl-CoA (≥93%), butyryl-CoA (≥90%), hexanoyl-CoA (≥85%), octanoyl-CoA, iso-valeryl CoA (≥90%), olivetol and sodium hexnoate were purchased from Sigma-Aldrich (Rehovot, Israel). Δ⁹-THCA was purchased from Silicol Scientific Equipment Ltd. (Or Yehuda, Israel). Acetic-D₃ acid (D>99%), propionic-D₅ acid (D>99%), butyric-D₅ acid (D>98%), pentanoic-Do acid (D>98%), heptanoic-D₅ acid (D>99%), octanoic-D₅ acid (D>99%), iso-butyric-D₇ acid (D>98%), ±2-methyl butyric-D₉ acid (D>99%), iso-valeric-D₉ acid (D>98%), iso-caproic-D₁₁ acid (D>98%) were purchased from C/D/N isotopes (Quebec, Canada). Phenylalanine-D₅ (D>98%) and phenylalanine-¹³C₉, ¹⁵N₁ (¹³C, ¹⁵N>99%) were synthesized by Cambridge Isotope Laboratories (Andover, MA). HeliCBGA (NP009525, 90%) was purchased from Analyticon Discovery GmbH (Potsdam, Germany). APHA was reported as an impurity (NP015136, 5%) in the heliCBGA analytical metabolite. OA (>90%), VA (>90%) and iso-butyryl-CoA were purchased from Cayman Chemical (Ann Arbor, MI, USA). PCP, naringenin chalcone, and pinocembrin chalcone were purchased from Wuhan ChemFaces Biochemical Co Ltd. (Hubei, China). Cinnamoyl-CoA and Coumaroyl-CoA were purchased from TransMIT GmbH (Hesse, Germany).

### LC-MS Chemical Analysis

Unless otherwise stated, 100 mg frozen powdered plant tissue were extracted with 300 μl ethanol, sonicated for 15 min, agitated for 30 min, and centrifuged at 14,000 g for 10 min. The supernatant was filtered through a 0.22 μm syringe filter and analyzed in the obtained concentration. Detection was performed using both targeted and non-targeted approaches as described in the Supplementary Text using an ultrahigh-performance liquid chromatography-tandem quadrupole time-of-flight (UPLC-qTOF) system comprised of a UPLC (Waters Acquity) with a diode array detector connected either to a XEVO G2-S QTof (Waters) or to Synapt HDMS (Waters). The chromatographic separation was performed on a 100 mm×2.1 mm i.d. (internal diameter), 1.7 μm UPLC BEH C18 column (Waters Acquity). The mobile phase consisted of 0.1% formic acid in acetonitrile:water (5:95, v/v; phase A) and 0.1% formic acid in acetonitrile (phase B). Terpenophenols were analyzed using UPLC Method 1 as follows: Initial conditions were 40% B for 1 min, raised to 100% B until 23 min, held at 100% B for 3.8 min, decreased to 40% B until 27 min, and held at 40% B until 29 min for re-equilibration of the system. The flow rate was 0.3 ml min⁻¹, and the column temperature was kept at 35° C. Intermediates and glucosylated metabolites were analyzed using UPLC Method 2 as follows: Initial conditions were from 0% to 28% B over 22 min, raised to 100% B until 36 min, held at 100% B for 2 min, decreased to 0% B until 38.5 min, and held at 40% B until 40 min for re-equilibration of the system. The flow rate was 0.3 ml min⁻¹, and the column temperature was kept at 35° C. Electrospray ionization (ESI) was used in either positive or negative ionization modes at an m/z range of 50-1,000 Da. Masses were detected with the following settings: capillary 1 kV, source temperature 140° C., desolvation temperature 450° C., and desolvation gas flow 800 1 h⁻¹. Argon was used as the collision gas. The MS system was calibrated with sodium formate and Leu encephalin was used as the lock mass. Data acquisition for untargeted analysis was performed in negative ionization using the MS^{E} mode. The collision energy was set to 4 eV for the low-energy function and to 15-50 eV ramp for the high-energy function. The R package Miso was run as previously described³⁴. Differential metabolites were selected if the fold change was greater or equal to 10 and the p-value was less than 0.05. MS/MS experiments were performed in positive or negative ionization modes according to the specific protonated or deprotonated masses with following settings: capillary spray of 1 kV; cone voltage of 30 cV; collision energy ramps were 10-45 eV for positive mode and 15-50 eV for negative mode.

Transient Expression of Selected Genes in N. benthamiana

Overexpression constructs of GFP (as negative control), CsOLS and CsOAC were generated using GoldenBraid cloning as described by Jozwiak et al. (2020) to a final vector of pAlpha2-Ubq10-CCD-Ter10. HuCoAT6, HuTKS4, and HuCBGAS were amplified and cloned in pAlpha2-NPT II-Ubq10-CCD-Ter10 vector digested with BsaI using ClonExpress II One Step Cloning kit (Vazyme). The full list of oligonucleotides used for cloning can be found in Supplementary Table 22. All plasmids were sequenced and transformed into Agrobacterium tumefaciens strain GV3101 by electroporation. A. tumefaciens harboring the overexpression constructs were grown overnight at 28° C. in Luria-Bertani (LB) medium in the presence of kanamycin and gentamycin. Bacterial cells were collected by centrifugation, washed, and resuspended in infiltration buffer (10 mM MES, 2 mM MgCl2, 2 mM Na3PO4, 0.5% glucose and 100 mM acetosyringone) to OD600=0.3. Equal volumes of A. tumefaciens suspension with different expression vectors were combined to obtain the desired gene combinations and incubated for 2 h at room temperature. The solutions were infiltrated into 4- or 5-week-old N. benthamiana leaves from the abaxial side using a 1-ml needleless syringe. Substrates (0.5 mM each) were infiltrated into the same leaf areas 2 days after initial infiltration, and leaves were collected for metabolite analysis after 24 h. Leaf samples were flash frozen and extracted as previously described with 300 μl methanol and analyzed on a similar UPLC system connected to an Orbitrap IQ-X Tribrid MS (Thermo Scientific, Bremen, Germany) using UPLC Method 2 in negative mode. The source parameters were: sheath gas flow rate, auxiliary gas flow rate and sweep gas flow rate: 45, 10 and 1 arbitrary units, respectively; vaporizer temperature: 300° C.; ion transfer tube temperature: 275° C.; spray voltage: 2.3 kV. The instrument was operated in full MS¹ with data dependent MS/MS (MS-dd-MS²). Data acquisition in full MS¹ mode was 60,000 resolution, the scan range 100-1,000 m/z, normalized automatic gain control (AGC) target of 25% and a maximum injection time (IT) of 50 ms. Data acquisition in dd-MS² mode was with 15,000 resolution, a normalized AGC target of 20%, maximum IT of 150 ms, isolation window of 1.5 m/z and normalized collision energy of 40. Identification of metabolites was performed using analytical standards and/or products from in vitro UGT enzyme assays.

### Example 1

### In Vivo Reconstruction of the Core Cannabinoid Pathway in a Heterologous Plant System

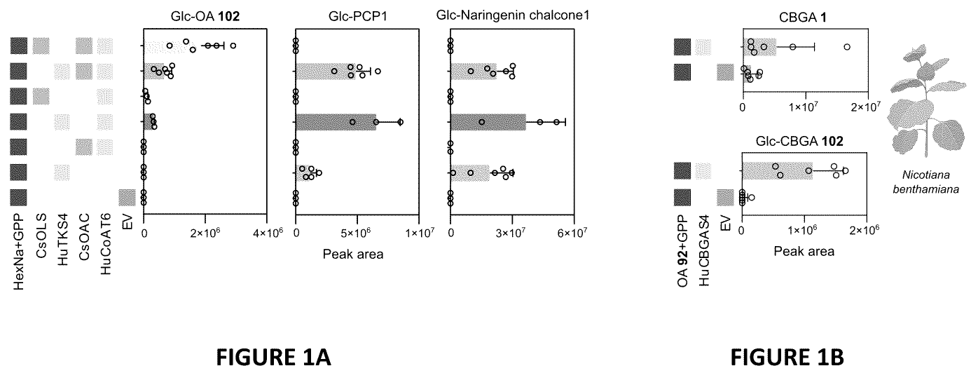

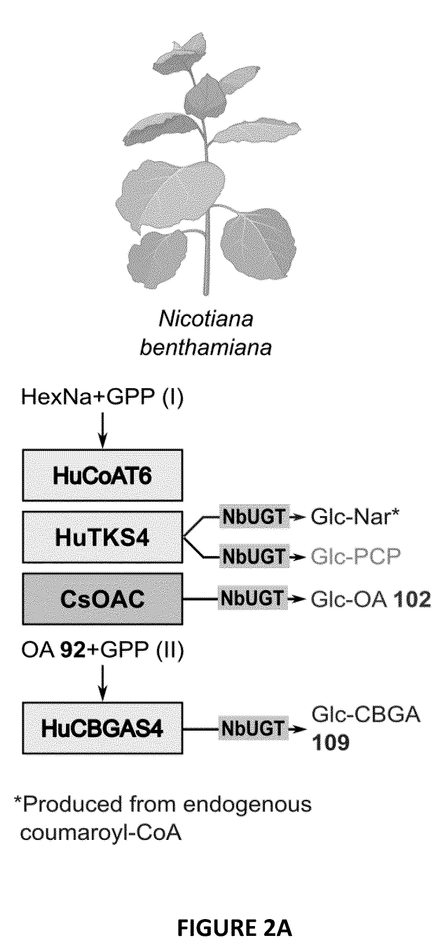

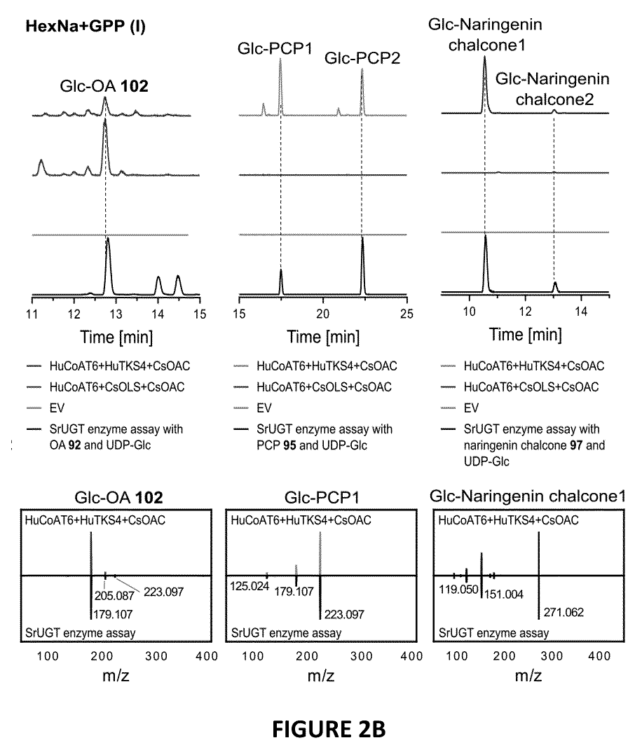

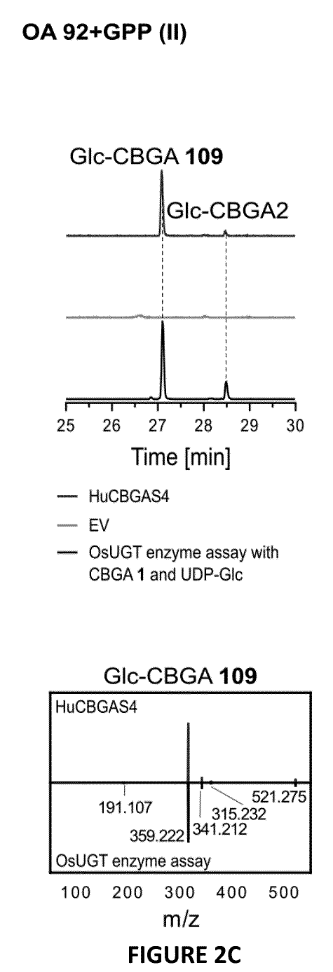

The inventors verified the in planta activity of enzymes towards CBGA by transiently co-expressing different combinations of Helichrysum endogenous genes related to cannabinoidogenesis, such as HuCoAT6, HuTKS4, and HuCBGAS4, and the Cannabis CsOAC and CsOLS in N. benthamiana leaves. Following leaves infiltration with sodium hexanoate and GPP, the inventors observed the production of glycosylated forms of OA (HuTKS4+CsOAC or CsOLS+CsOAC) and PCP (only with HuTKS4, FIGS. 1A and 2A-2B). This was consistent with previous studies reporting OA glycosylation by endogenous enzymes in this plant. Interestingly, the inventors also observed glycosylated products of naringenin chalcone with HuTKS4, suggesting that this enzyme can accept aromatic substrates in addition to aliphatic types (FIGS. 1A and 2A-2B). In this regard, the inventors did not observe CBGA or its glycosylated forms with HuCBGAS4, possibly due to the low availability of OA and its rapid glycosylation in planta. When leaves expressing HuCBGAS4 were infiltrated with OA and GPP, CBGA and Glc-CBGA were observed (FIGS. 1B, 2A, and 2C).

Therefore, the inventors conclude that a plant comprising Helichrysum endogenous genes encoding cannabinoidogenesis-related proteins/enzymes as well as one or more genes encoding cannabinoidogenesis-related proteins/enzymes derived from Cannabis sativa cannabinoids or precursor(s) thereof, is suitable for production of at least one cannabinoid or a precursor thereof, particularly a transgenic Helichrysum cell, tissue, or plant comprising one or more transgenes encoding(g) cannabinoidogenesis-related protein(s)/enzyme(s) derived from Cannabis sativa, Further, the transgenic cell, tissue, or plant may also be suitable for production of at least one acyl phloroglucinoid, amorfrutin (cannabinoid-like), prenyl chalcone, a precursor thereof, or any combination thereof.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A transgenic Helichrysum umbraculigerum (Less.) cell, tissue, or plant, comprising an artificial DNA molecule comprising a first nucleic acid sequence selected form the group consisting of:
a. SEQ ID NO: 1 or a functional analog thereof having at least 80% sequence homology to SEQ ID NO: 1;
b. SEQ ID NO: 2 or a functional analog thereof having at least 80% sequence homology to SEQ ID NO: 2;
c. SEQ ID NO: 3 or a functional analog thereof having at least 80% sequence homology to SEQ ID NO: 3;
d. SEQ ID NO: 4 or a functional analog thereof having at least 80% sequence homology to SEQ ID NO: 4;
e. SEQ ID NO: 5 or a functional analog thereof having at least 80% sequence homology to SEQ ID NO: 5;
f. SEQ ID NO: 6 or a functional analog thereof having at least 80% sequence homology to SEQ ID NO: 6;
g. SEQ ID NO: 7 or a functional analog thereof having at least 80% sequence homology to SEQ ID NO: 7;
h. SEQ ID NO: 8 or a functional analog thereof having at least 80% sequence homology to SEQ ID NO: 8; and
i. any combination of (a) to (h).

2. The transgenic H. umbraculigerum cell, tissue, or plant, of claim 1, wherein said artificial DNA molecule comprises a first nucleic acid sequence comprising SEQ ID NO: 3 and SEQ ID NO: 4, or a functional analog thereof having at least 80% sequence homology thereto.

3. The transgenic H. umbraculigerum cell, tissue, or plant, of claim 1, wherein said artificial DNA molecule further comprises at least one promoter for transcription in a plant cell, and optionally wherein: (i) said at least one promoter is operably linked to any one of: said SEQ ID Nos: 1-8; (ii) said at least one promoter is a constitutive promoter or an inducible promoter, or both (i) and (ii).

4.-5. (canceled)

6. The transgenic H. umbraculigerum cell, tissue, or plant, of claim 1, wherein said artificial DNA molecule further comprises a second nucleic acid sequence encoding at least one protein or enzyme related to cannabinoid synthesis or regulation thereof.

7. An extract or a fraction thereof, derived from the transgenic H. umbraculigerum cell, tissue, and plant, of claim 1, and optionally wherein said extract or a fraction thereof comprises at least one cannabinoid, a precursor thereof, or any combination thereof.

8. (canceled)

9. A composition comprising the extract or a fraction thereof of claim 7, and an acceptable carrier.

10. A method for producing the transgenic H. umbraculigerum cell or tissue of claim 1, comprising providing a H. umbraculigerum cell or tissue and introducing said artificial DNA molecule comprising said first nucleic acid sequence selected from the group consisting of: SEQ ID Nos: 1-8, a functional analog having at least 80% thereto, and any combination thereof, to said cell or tissue, thereby producing the transgenic H. umbraculigerum cell or tissue.

11. The method of claim 10, wherein said artificial DNA molecule comprises a first nucleic acid sequence comprising SEQ ID NO: 3 and SEQ ID NO: 4, or a functional analog thereof having at least 80% sequence homology thereto.

12. The method of claim 10, further comprising a step of regenerating said transgenic H. umbraculigerum cell or tissue into a plant, thereby producing a transgenic H. umbraculigerum plant.

13. The method of claim 10, wherein said cell or tissue are obtained or derived from a transformable explant, and optionally wherein said transformable explant is selected from the group consisting of: a callus, an embryo, and a cell suspension.

14. The method of claim 13, wherein said transformable explant is selected from the group consisting of: a callus, an embryo, and a cell suspension.

15. The method of claim 10, wherein said artificial DNA molecule is a plasmid or an agrobacterium comprising said first nucleic acid sequence.

16. The method of claim 10, wherein said artificial DNA molecule further comprises a second nucleic acid sequence encoding at least one protein or enzyme related to cannabinoid synthesis or regulation thereof.

17. The method of claim 10, wherein any one of said transgenic H. umbraculigerum cell, tissue, and plant, is characterized by being capable of synthesizing at least one cannabinoid, a precursor thereof, or both.

18. A method for producing a cannabinoid or a precursor thereof, comprising:
a. providing a transgenic H. umbraculigerum cell, tissue, or plant, comprising an artificial DNA molecule comprising a first nucleic acid sequence selected from the group consisting of: SEQ ID Nos: 1-8, a functional analog having at least 80% thereto, and any combination thereof; and
b. culturing said transgenic H. umbraculigerum cell, tissue, or plant, such that a cannabinoid or a precursor thereof is produced,
thereby producing the cannabinoid or a precursor thereof.

19. The method of claim 18, wherein said artificial DNA molecule comprises a first nucleic acid sequence comprising SEQ ID NO: 3 and SEQ ID NO: 4, or a functional analog thereof having at least 80% sequence homology thereto, and optionally wherein said method further comprises a step proceeding step (b) comprising extracting said cannabinoid or a precursor thereof from said cultured transgenic H. umbraculigerum cell, tissue, or plant.

20. (canceled)

21. The method of claim 19, wherein: (i) said extracting comprises extracting a tissue derived from said transgenic H. umbraculigerum plant comprising a chlorophyll, a trichome, or both; and (ii) said extracting comprises extracting a stem, a leaf, a portion thereof, or any combination thereof, of said transgenic H. umbraculigerum plant; or (iii) both (i) and (ii).

22. (canceled)

23. A cannabinoid or a precursor thereof, produced according to the method of claim 18.

24. An extract comprising a cannabinoid or a precursor thereof, obtained according to the method of claim 19.

25. A composition comprising the cannabinoid or a precursor thereof of claim 23, and an acceptable carrier.
